(19) United States
(12) Patent Application Publication
(10) Pub. No.: US 2024/0192388 A1
(43) Pub. Date: Jun. 13, 2024

(54) CT DETECTOR

(71) Applicant: SHANGHAI UNITED IMAGING HEALTHCARE CO., LTD., Shanghai (CN)

(72) Inventor: YAN-YAN LIU, Shanghai (CN)

(21) Appl. No.: 18/537,652

(22) Filed: Dec. 12, 2023

(30) Foreign Application Priority Data
Dec. 13, 2022 (CN) 202223341497.2

(51) Int. Cl. G01T 1/20; A61B 6/03; A61B 6/42

(52) U.S. Cl. CPC G01T 1/2018; A61B 6/032; A61B 6/4266

(57) ABSTRACT

The present disclosure provides a computed tomography (CT) detector, including at least two detection modules and a ray absorber. A gap is formed between each two adjacent detection modules. The ray absorber is arranged in the gap or arranged at an X-ray-incidence end of the gap. The ray absorber is made of a material of high atomic number.

### CROSS-REFERENCE TO RELATED APPLICATION

The present application claims the priority of Chinese patent application No. 202223341497.2, filed on Dec. 13, 2022, and entitled “CT DETECTOR”, which is incorporated herein by reference in its entirety.

### TECHNICAL FIELD

The present disclosure relates to the field of computed tomography (CT) imaging technology, and in particular to a CT detector.

### BACKGROUND

Existing CT imaging equipment constructs images by using transmission rays (beams). The transmission rays are generated by X-rays. The X-rays, after passing through a human body or an object, generate transmission rays and scattered rays. The scattered rays are disadvantageous to image reconstruction and need to be eliminated. The X-rays irradiating the detector may be converted into electrical signals by the detector which is the most important part of the equipment. The detector guides the X-rays radiating from the ray generator to corresponding pixel units in detection modules. Referring to FIG. 1, the detectors are assembled manually in the form of modules. Inside gaps between the pixel units in one detection module are small and have identical dimensions. However, there are inevitably some gaps formed between the detection modules, and these gaps are large and dimensions thereof are quite different. Due to the existence of these gaps between the detection modules, some X-rays, mainly scattered X-rays, may pass through the gaps and enter the pixel units close to the gaps. In particular, the pixel units proximate to the gaps between the detection modules are more affected, which will cause the pixel units proximate to the gaps between the detection modules to receive more X-rays than other pixel units do, so that the scattering distribution presents a high-frequency scattering distribution. In the final reconstructed image, the pixel units of the detector, which are proximate to the gaps between the detection modules, may easily generate artifacts, thus affecting the final reconstructed image.

### SUMMARY

The present disclosure provides a CT detector. The CT detector includes at least two detection modules and a ray absorber. A gap is formed between each two adjacent detection modules. The ray absorber is arranged in the gap or arranged at an X-ray-incidence end of the gap. The ray absorber is made of a material of high atomic number.

In some of the embodiments, the ray absorber is arranged at the X-ray-incidence end of the gap and disposed on the two adjacent detection modules.

In some of the embodiments, a projection of the ray absorber along X-rays directions covers the gap.

In some of the embodiments, a first dimension of the ray absorber is greater than or equal to a width of the gap, wherein the width of the gap is a distance between the two adjacent detection modules, and the first dimension of the ray absorber is a dimension in the same direction as the width of the gap.

In some of the embodiments, a second dimension of the ray absorber is greater than or equal to a length of the gap, wherein the length of the gap is a dimension of the gap in a direction perpendicular to the vertical section of the ray absorber, and the second dimension of the ray absorber is a dimension in the direction perpendicular to the vertical section. The vertical section is obtained from a cutting by an intersecting plane, wherein the intersecting plane is vertically perpendicular to the two adjacent detection modules and passes through respective central planes of the two adjacent detection modules.

In some of the embodiments, the first dimension of the ray absorber is greater than or equal to a distance between two pixel units proximate to two edges of the gap respectively.

In some of the embodiments, a vertical section of the ray absorber is in a circular shape. The first dimension of the ray absorber is a diameter of the vertical section of the ray absorber.

In some of the embodiments, a vertical section of the ray absorber is in a rectangular shape.

In some of the embodiments, the ray absorber is arranged at an upstream position of the X-ray-incidence end of the gap, and the upstream position of the X-ray-incidence end is a position where the X-rays pass to radiate towards the gap.

In some of the embodiments, a region shielded by the ray absorber includes a first shielded region and a second shielded region. The first shielded region is a range of the gap between the two adjacent detection modules, and the second shielded region is a minimum range from two edges of the gap to respective adjacent pixel units.

In some of the embodiments, a first dimension of the ray absorber is configured based on a first distance, a second distance, and the shielded region. The first distance is a distance between the ray absorber and a surface of one of the two adjacent detection modules, and the second distance is a distance from a focus of the X-rays to a surface of the one of the two adjacent detection modules. The first dimension of the ray absorber is a dimension in the same direction as a width of the gap, and the width of the gap is a distance between the two adjacent detection modules.

In some of the embodiments, the ray absorber is an integral component arranged in the gap between the two adjacent detection modules.

In some of the embodiments, the ray absorber is in a cuboid shape. A first dimension of the ray absorber is equal to a width of the gap. The width of the gap is a distance between the two adjacent detection modules, and the first dimension of the ray absorber is a dimension in the same direction as the width of the gap.

In some of the embodiments, the ray absorber is in a cuboid shape, a vertical section of the ray absorber is in a rectangular shape, and the vertical section is obtained from a cutting by an intersecting plane, the intersecting plane is vertically perpendicular to the two adjacent detection modules and passes through respective central planes of the two adjacent detection modules, and a second dimension of the ray absorber is equal to a length of the gap, the length of the gap is a dimension of the gap in a direction perpendicular to a vertical section of the ray absorber, and the second dimension of the ray absorber is a dimension in the direction perpendicular to the vertical section.

In some of the embodiments, the ray absorber is in a cuboid shape, a vertical section of the ray absorber is in a rectangular shape, and the vertical section is obtained from a cutting by an intersecting plane, the intersecting plane is vertically perpendicular to the two adjacent detection modules and passes through respective central planes of the two adjacent detection modules, and a third dimension of the ray absorber is equal to a height of the gap. The third dimension of the ray absorber is a dimension of the ray absorber along a vertical direction and in the vertical section, and the height of the gap is a dimension of the gap along the vertical direction and in the vertical section.

In some of the embodiments, the ray absorber includes a first sub-ray absorber and a second sub-ray absorber, and the first sub-ray absorber and the second sub-ray absorber are arranged on two side walls of the gap.

In some embodiments, the first sub-ray absorber and the second sub-ray absorber are spaced apart.

In some of the embodiments, a width of the first sub-ray absorber and a width of the second sub-ray absorber are both less than a distance between the two adjacent detection modules, and the width of the first sub-ray absorber and the width of the second sub-ray absorber are respective dimensions of the first sub-ray absorber and second sub-ray absorber in the same direction as a width of the gap, and the width of the gap is a distance between the two adjacent detection modules.

In some of the embodiments, a minimum width of the first sub-ray absorber and a minimum width of the second sub-ray absorber are determined based on the atomic number of the material of high atomic number.

In some of the embodiments, the atomic number of the material of high atomic number is greater than or equal to an atomic number of aluminum.

In some of the embodiments, the CT detector further includes slideways arranged on the two adjacent detection modules respectively, and the ray absorber is movably arranged on the slideways.

The present invention can achieve beneficial effects as follows. In this invention, the ray absorber made of the material of high atomic number shields the gap between the two adjacent detection modules, thereby effectively restraining a high-frequency scattering during the scan performed by the computed tomography imaging equipment, thereby avoiding the occurrence of the artifacts in the final reconstructed image.

### BRIEF DESCRIPTION OF THE DRAWINGS

In order to illustrate the technical solutions in the embodiments of the present application or in the prior art more clearly, the drawings needed to be used for the description of the embodiments or for the description of the prior art will be briefly introduced hereinafter. Obviously, the attached drawings in the following description are just embodiments of the present application. For those of ordinary skill in the art, other drawings may be obtained based on the disclosed drawings without creative efforts.

FIG. 1 is a schematic vertical section view showing a structure of a CT detector in related art.

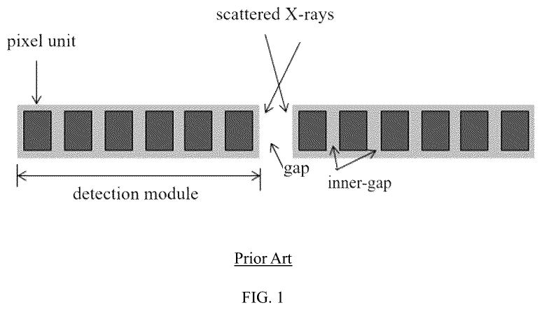

FIG. 2 is a block diagram showing a structure of the CT detector according to an embodiment of the present disclosure.

FIG. 3 is a schematic vertical section view showing a structure of the CT detector according to an embodiment of the present disclosure, where a ray absorber is in a spherical shape.

FIG. 4 is a schematic vertical section view showing a structure of the CT detector according to an embodiment of the present disclosure, where the ray absorber is in a cuboid shape.

FIG. 5 is a schematic vertical section view showing a structure of the CT detector according to an embodiment of the present disclosure, where the ray absorber is arranged to be away from detection modules.

FIG. 6 is a schematic vertical section view showing a structure of the CT detector according to an embodiment of the present disclosure, where the ray absorber is arranged in the gap between two adjacent detection modules.

FIG. 7 is a schematic vertical section view showing a structure of the CT detector according to an embodiment of the present disclosure, where two cuboid sub-ray absorbers are arranged to cover side walls of two adjacent detection modules, respectively.

### DETAILED DESCRIPTION OF THE EMBODIMENTS

In order to make the present application to be understood, the present application will be described more comprehensively hereinafter with reference to the related drawings. The embodiments of the present application are shown in the attached drawings. However, the present application may be implemented in various forms and is not limited to the embodiments described herein. On the contrary, these embodiments are provided to make disclosed contents of the present application to be understood thoroughly and comprehensively.

In the structure of the CT detector in the related art, the detector is assembled in the form of modules. Pixel units are arranged in the detection module, and configured to receive X-rays and output response data. The inner-gaps between the pixel units in the same detection module are very small, and the dimensions of these inner-gaps are consistent. Therefore, when a CT scan is performed, the differences between strengths of scattered rays received by the pixel units are small, which has a small impact on the final reconstructed image. However, the gaps between each two adjacent detection modules are large, and the dimensions of the gaps between each two detection modules are not identical and fluctuate greatly for the reason that detection modules are usually assembled manually, which causes the strengths of the scattered rays received by the photosensitive areas proximate to the gaps between each two adjacent detection modules to present a high-frequency scattering distribution. In the final reconstructed image, annular artifacts will be generated at the edges of the detection modules proximate to the gaps between each two adjacent detection modules.

Based on the defects above, the present disclosure provides a CT detector to solve the technical problem that, in the prior art, the pixel units proximate to the gaps between each two adjacent modules generate artifacts in the final reconstructed image, because when X-rays scatter at the edge of the detector, the pixel units proximate to the gaps between each two adjacent detection modules receive more rays than other pixel units do due to the existence of the gaps between each two adjacent detection modules.

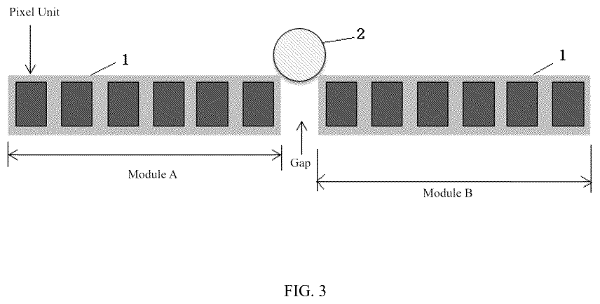

An embodiment of the present disclosure provides a CT detector. Referring to FIG. 3 to FIG. 5, the CT detector includes at least two detection modules 1 (namely, a module A and a module B) and a ray absorber 2. A gap is formed between each two adjacent detection modules 1. The ray absorber 2 is arranged at an X-ray-incidence end of the gap The X-ray-incidence end is an end of the gap which the X-rays irradiate. The ray absorber 2 is made of a material of high atomic number. The ray absorber 2 is configured to absorb the X-rays radiating towards the gap.

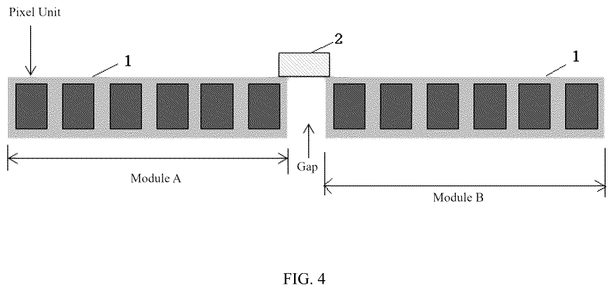

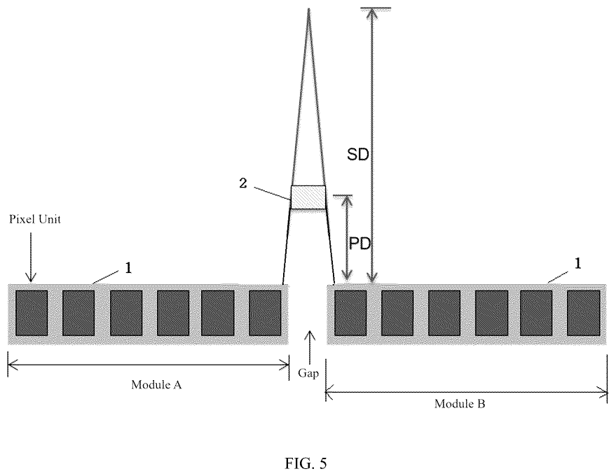

As shown in FIG. 3 and FIG. 4, the ray absorber 2 is arranged at the X-ray-incidence end of the gap and disposed on the two adjacent detection modules 1, namely the ray absorber 2 may be in contact with the two adjacent detection modules 1 at the same time. As shown in FIG. 5, the ray absorber 2 may not be in contact with the detection modules 1, but be arranged at an upstream position of the X-ray-incidence end of the gap between the two adjacent detection modules 1, that is, the ray absorber 2 is arranged at a position through which the X-rays pass to radiate towards the gap.

In this embodiment, the ray absorber 2 made of the material of high atomic number is arranged to shield the gap between the two adjacent detection modules 1, thereby effectively restraining high-frequency scattering during a scan performed by the computed tomography equipment, and further avoiding annular artifacts generated in the final reconstructed image.

In some embodiments, the ray absorber is configured such that a projection of the ray absorber along the X-rays directions covers the gap. In some embodiments, as shown in FIG. 3, the ray absorber 2 is in a spherical shape, and a vertical section of the ray absorber 2 is in a circular shape. The vertical section is obtained from a cutting by an intersecting plane, wherein the intersecting plane is vertically perpendicular to the two adjacent detection modules and passes through respective central planes of the two adjacent detection modules. In some embodiments, as shown in FIG. 4, the ray absorber 2 is in a cuboid shape. A region shielded by the ray absorber 2 includes a first shielded region and a second shielded region. The first shielded region is a range of the gap between two adjacent detection modules 1, and the second shielded region is a minimum range from the two edges of the gap to respective adjacent pixel units, that is, the minimum range from the two edges of the gap to effective photosensitive areas of the two adjacent detection modules 1 respectively.

In some embodiments, as shown in FIG. 5, the ray absorber 2 is arranged at the upstream position of the gap between the two adjacent detection modules 1, for the reason that, in an actual implementation, it may be difficult to arrange the ray absorber 2 to be in tight contact with the two adjacent detection modules 1. In addition, because the X-rays radiate divergently from a focus to the photosensitive areas of the detection modules 1, when the ray absorber 2 is arranged away from the two adjacent detection modules 1, the first dimension of the ray absorber 2 may be reduced accordingly. A first dimension of the ray absorber 2 is configured according to a first distance, a second distance, and the shielded range. The first distance is a distance PD between the ray absorber 2 and a surface of one of the two adjacent detection modules 1, and the second distance is a distance SD from the focus of the X-ray source to a surface of the one of the two adjacent detection modules 1.

Referring to FIG. 5, the width Wid_0 of the ray absorber 2 is calculated as follows:

Wid_0=Wid×(SD−PD)/SD

where SD denotes the distance from the focus of the X-ray to the surface of the one of the two adjacent detection modules 1, PD denotes the distance between the ray absorber 2 and the one of the two adjacent detection modules 1, and Wid denotes the distance between two pixel units proximate to the two edges of the gap respectively. The first direction of the ray absorber 2 is configured such that not only the shielding effects of the ray absorber 2 are ensured, but also the production cost of the ray absorber 2 is reduced relatively. In some embodiments, the vertical section of the ray absorber 2 is in a circular shape, and the first dimension of the ray absorber 2 is a diameter of the vertical section of the ray absorber 2.

In some embodiments, the vertical section of the ray absorber 2 may also be L-shaped, T-shaped, or may be in any other shape. The shape of the ray absorber 2 is configured according to the dimensions of the detection module 1 and the dimensions of the gap between the two adjacent detection modules 1, thereby ensuring the shielding effects of the ray absorber 2.

In some embodiments, in order to absorb the X-rays radiating towards the gap as many as possible, the diameter of the ray absorber 2 having a circular section is greater than or equal to the width of the gap (namely, a distance between the two adjacent detection modules 1 in the figures), and is greater than or equal to a length of the gap. The length of the gap refers to a dimension of the gap in a direction perpendicular to the vertical section. In one of the embodiments, the diameter of the ray absorber 2 is equal to the distance between two pixel units proximate to the two edges of the gap respectively.

In an embodiment, the atomic number of the material of high atomic number is greater than or equal to the atomic number of aluminum.

For example, the material of high atomic number includes tungsten, molybdenum, high-density material, or high-density alloy, etc.

In this embodiment, the ray absorber 2 is made of high energy-absorbing material, which ensures that the ray absorber 2 can effectively restrain the high-frequency scattering and avoid an occurrence of annular artifacts in the final reconstructed image.

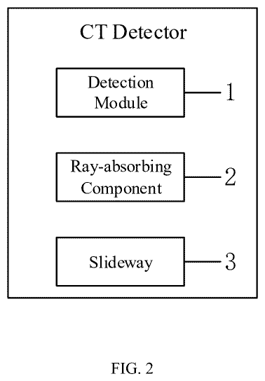

In an embodiment, as shown in FIG. 2, the CT detector further includes slideways 3. The slideways 3 each are arranged on the two adjacent detection modules 1, so that the ray absorber 2 may be movably arranged on the slideways 3.

In this embodiment, the ray absorber 2 is movably arranged on the slideway 3, so as to accurately locate an installation position of the ray absorber 2, thereby accurately locating a ray-absorbing range, and improving the installation efficiency of the ray absorber 2.

In an embodiment of the CT detector of the present disclosure, the CT detector includes at least two adjacent detection modules 1 (namely, the module A and the module B) and the ray absorber 2. The gap is formed between the two adjacent detection modules 1. The ray absorber 2 is in contact with the two adjacent detection modules 1, and the ray absorber 2 is arranged in the gap. The ray absorber 2 is made of a material of high atomic number. The ray absorber 2 is configured to absorb the X-rays radiating towards the gap.

In this embodiment, the ray absorber 2 is made of the material of high atomic number and shields the side walls of the gap between the two adjacent detection modules 1, thereby effectively restraining the high-frequency scattering during a scan performed by the computed tomography imaging equipment, and further avoiding the occurrence of the annular artifacts in the final reconstructed image.

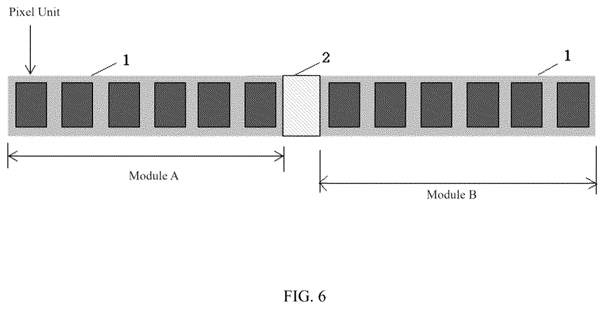

In some embodiments, referring to FIG. 6, FIG. 6 is a schematic vertical section view showing a structure of the CT detector according to an embodiment of the present disclosure, where the ray absorber is arranged in the gap between two adjacent detection modules. In the structure shown in FIG. 6, the X-rays may enter the area of the gap, but cannot enter the photosensitive areas of the pixel units of the detection modules proximate to the gap through side walls of the gap, thus achieving the shielding effects.

In this implementation, the ray absorber 2 is an integral component. In order to ensure the shielding effects of the ray absorber 2, it is only necessary to configure the ray absorber 2 according to the dimensions of the gap between the two adjacent detection modules 1, thus reducing the difficulties in producing the ray absorber 2.

In some embodiments, the vertical section of the ray absorber 2 is rectangular, circular, L-shaped, T-shaped, or may be in any other shape. The shape of the ray absorber 2 is configured according to the dimensions of the detection module 1 and the dimensions of the gap between the two adjacent detection modules 1, thereby ensuring the shielding effects of the ray absorber 2.

In some embodiments, as shown in FIG. 6, the ray absorber 2 is in the shape of a cuboid, and a vertical section of the ray absorber is in a rectangular shape. A first dimension of the ray absorber 2 is equal to the width of the gap, namely the distance between the two adjacent detection modules. A second dimension of the ray absorber 2 is greater than or equal to the length of the gap, and a third dimension of the ray absorber 2 is equal to a height of the gap. The length of the gap is a dimension of the gap in a direction perpendicular to the vertical section of the ray absorber, and the second dimension of the ray absorber is a dimension in the direction perpendicular to the vertical section. The third dimension of the ray absorber is a dimension of the ray absorber along a vertical direction, and in the vertical section, and the height of the gap is a dimension of the gap along the vertical direction and in the vertical section.

In an embodiment, the atomic number of the material of high atomic number is greater than or equal to the atomic number of aluminum.

For example, the material of high atomic number includes tungsten, molybdenum, high-density material, or high-density alloy, etc.

In this embodiment, the ray absorber 2 is made of the material of the high atomic number, which ensures that the ray absorber 2 can effectively restrain the high-frequency scattering and avoid the occurrence of the annular artifacts in the final reconstructed image.

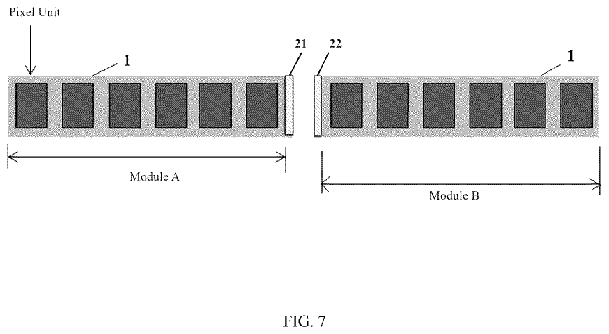

An embodiment of the present disclosure provides a CT detector. As shown in FIG. 7, the CT detector includes at least two adjacent detection modules 1 (namely, the module A and the module B) and the ray absorber 2. The gap is formed between the two adjacent detection modules 1. The ray absorber 2 is in contact with the two adjacent detection modules 1 and arranged in the gap. The ray absorber 2 includes a first sub-ray absorber 21 and a second sub-ray absorber 22. The first sub-ray absorber 21 and the second sub-ray absorber 22 are arranged on two side walls of the gap respectively. The ray absorber 2 is made of a material of high atomic number. The ray absorber 2 is configured to absorb the scattered X-rays radiating towards the two side walls of the gap.

In this embodiment, the ray absorber 2 is made of the material of high atomic number and shields the side walls of the gap between the two adjacent detection modules 1, thereby effectively restraining the high-frequency scattering during the scan performed by the computed tomography imaging equipment, and further avoiding the occurrence of the annular artifacts in the final reconstructed image.

In some embodiments, the first sub-ray absorber 21 and the second sub-ray absorber 22 each are in a shape of a cuboid. The length of the first sub-ray absorber 21 and the length of the second sub-ray absorber 22 are equal to the length of the gap, and the height of the first sub-ray absorber 21 and the height of the second sub-ray absorber 22 are equal to the height of the gap. The length of the first sub-ray absorber 21 and the length of the second sub-ray absorber 22 are dimensions along the direction perpendicular to the vertical section. The height of the first sub-ray absorber 21 and the height of the second sub-ray absorber 22 are dimensions of the first sub-ray absorber 21 and the second sub-ray absorber 22 along the vertical direction and in the vertical section.

If the material of high atomic number is filled in the gap, as shown in FIG. 7, the entire gap area does not need to be fully filled with the ray absorber 2, and only the side walls of the detection modules 1 need to be covered with the first sub-ray absorber 21 and the second sub-ray absorber 22, respectively. The X-rays may enter the area of the gap, but cannot enter the photosensitive areas of the pixel units of the detection modules proximate to the gap through the side walls of the gap, thus achieving the shielding effects.

In this embodiment, the first sub-ray absorber 21 and the second sub-ray absorber 22 reduce the difficulties in producing the ray absorber 2 and ensure the shielding effects of the ray absorber 2.

In some embodiments, the first sub-ray absorber 21 and the second sub-ray absorber 22 are spaced apart. In some embodiments, a width of the first sub-ray absorber 21 and a width of the second sub-ray absorber 22 are both less than the distance between the two adjacent detection modules. A minimum width of the first sub-ray absorber 21 and a minimum width of the second sub-ray absorber 22 are determined according to the atomic number of the material of high atomic number. The larger the atomic number is, the smaller the minimum width of the first sub-ray absorber 21 and the minimum width of the second sub-ray absorber 22 are.

In an embodiment, the atomic number of high atomic number material is greater than or equal to the atomic number of aluminum.

For example, the material of high atomic number includes tungsten, molybdenum, high-density material, or high-density alloy, etc.

In this embodiment, the first sub-ray absorber 21 and the second sub-ray absorber 22 are made of the material of high atomic number, which can effectively restrain the high-frequency scattering and avoid the occurrence of the annular artifacts in the final reconstructed image.

What is described above are some embodiments of the present disclosure. It should be understood for those skilled in the art that these embodiments are merely examples for illustration, and that the protection scope of the present disclosure is defined by the appended claims. For those skilled in the art, various changes or modifications may be made based on these embodiments without departing from the principles and essences of the present disclosure, but these changes or modifications all fall within the protection scope of the present disclosure.

What is claimed is:

1. A computed tomography (CT) detector, comprising:
at least two detection modules, a gap being formed between each two adjacent detection modules; and
a ray absorber arranged in the gap or arranged at an X-ray-incidence end of the gap, wherein the ray absorber is made of a material of high atomic number.

2. The CT detector of claim 1, wherein the ray absorber is arranged at the X-ray-incidence end of the gap and disposed on the two adjacent detection modules.

3. The CT detector of claim 2, wherein a projection of the ray absorber along X-rays directions covers the gap.

4. The CT detector of claim 3, wherein:
a first dimension of the ray absorber is greater than or equal to a width of the gap, wherein the width of the gap is a distance between the two adjacent detection modules, and the first dimension of the ray absorber is a dimension in the same direction as the width of the gap.

5. The CT detector of claim 4, wherein:
a second dimension of the ray absorber is greater than or equal to a length of the gap, wherein the length of the gap is a dimension of the gap in a direction perpendicular to a vertical section of the ray absorber, and the second dimension of the ray absorber is a dimension in the direction perpendicular to the vertical section; and
the vertical section is obtained from a cutting by an intersecting plane, wherein the intersecting plane is vertically perpendicular to the two adjacent detection modules and passes through respective central planes of the two adjacent detection modules.

6. The CT detector of claim 4, wherein the first dimension of the ray absorber is greater than or equal to a distance between two pixel units proximate to two edges of the gap respectively.

7. The CT detector of claim 5, wherein the vertical section of the ray absorber is in a circular shape; and
the first dimension of the ray absorber is a diameter of the vertical section of the ray absorber.

8. The CT detector of claim 5, wherein the vertical section of the ray absorber is in a rectangular shape.

9. The CT detector of claim 1, wherein the ray absorber is arranged at an upstream position of the X-ray-incidence end of the gap, and the upstream position of the X-ray-incidence end is a position where the X-rays pass to radiate towards the gap.

10. The CT detector of claim 9, wherein:
a region shielded by the ray absorber comprises a first shielded region and a second shielded region;
the first shielded region is a range of the gap between the two adjacent detection modules; and
the second shielded region is a minimum range from two edges of the gap to respective adjacent pixel units.

11. The CT detector of claim 2, wherein:
a first dimension of the ray absorber is configured based on a first distance, a second distance, and the shielded region;
the first distance is a distance between the ray absorber and a surface of one of the two adjacent detection modules;
the second distance is a distance from a focus of the X-rays to the surface of the one of the two adjacent detection modules; and
the first dimension of the ray absorber is a dimension in the same direction as a width of the gap, and the width of the gap is a distance between the two adjacent detection modules.

12. The CT detector of claim 1, wherein the ray absorber is an integral component arranged in the gap between the two adjacent detection modules.

13. The CT detector of claim 12, wherein:
the ray absorber is in a cuboid shape; and
a first dimension of the ray absorber is equal to a width of the gap, wherein the width of the gap is a distance between the two adjacent detection modules, and the first dimension of the ray absorber is a dimension in the same direction as the width of the gap.

14. The CT detector of claim 12, wherein:
the ray absorber is in a cuboid shape; a vertical section of the ray absorber is in a rectangular shape, and the vertical section is obtained from a cutting by an intersecting plane, wherein the intersecting plane is vertically perpendicular to the two adjacent detection modules and passes through respective central planes of the two adjacent detection modules; and a second dimension of the ray absorber is equal to a length of the gap, wherein the length of the gap is a dimension of the gap in a direction perpendicular to the vertical section of the ray absorber, and the second dimension of the ray absorber is a dimension in the direction perpendicular to the vertical section; or
the ray absorber is in a cuboid shape; a vertical section of the ray absorber is in a rectangular shape, and the vertical section is obtained from a cutting by an intersecting plane, wherein the intersecting plane is vertically perpendicular to the two adjacent detection modules and passes through respective central planes of the two adjacent detection modules; and a third dimension of the ray absorber is equal to a height of the gap, wherein the third dimension of the ray absorber is a dimension of the ray absorber along a vertical direction and in the vertical section, and the height of the gap is a dimension of the gap along the vertical direction and in the vertical section.

15. The CT detector of claim 1, wherein the ray absorber comprises a first sub-ray absorber and a second sub-ray absorber, and the first sub-ray absorber and the second sub-ray absorber are arranged on two side walls of the gap.

16. The CT detector of claim 15, wherein the first sub-ray absorber and the second sub-ray absorber are spaced apart.

17. The CT detector of claim 15, wherein a width of the first sub-ray absorber and a width of the second sub-ray absorber are both less than a distance between the two adjacent detection modules, and the width of the first sub-ray absorber and the width of the second sub-ray absorber are respective dimensions of the first sub-ray absorber and second sub-ray absorber in the same direction as a width of the gap, and the width of the gap is a distance between the two adjacent detection modules.

18. The CT detector of claim 17, wherein a minimum width of the first sub-ray absorber and a minimum width of the second sub-ray absorber are determined based on the atomic number of the material of high atomic number.

19. The CT detector of claim 1, wherein the atomic number of the material of high atomic number is greater than or equal to an atomic number of aluminum.

20. The CT detector of claim 1, further comprising slideways arranged on the two adjacent detection modules respectively, and the ray absorber is movably arranged on the slideways.
